(19) United States
(12) Patent Application Publication
(10) Pub. No.: US 2008/0177311 A1
(43) Pub. Date: Jul. 24, 2008

(54) FACET JOINT IMPLANT SIZING TOOL

(75) Inventors: Charles J. Winslow, Walnut Creek, CA (US); Steven T. Mitchell, Pleasant Hill, CA (US); Scott A. Yerby, Montara, CA (US)

Correspondence Address: HAYNES AND BOONE, LLP, 901 Main Street, Suite 3100, Dallas, TX, 75202, US

(73) Assignee: ST. FRANCIS MEDICAL TECHNOLOGIES, INC., Alameda, CA (US)

(21) Appl. No.: 11/554,401

(22) Filed: Oct. 30, 2006

(51) Int. Cl. A61B 17/56

(52) U.S. Cl. 606/248

(57) ABSTRACT

In an embodiment of the present invention, a tool resembles an implant for positioning within a cervical facet joint. The tool can be used for distracting and/or sizing the cervical facet joint and thereby distracting the cervical spine and increasing the area of the canals and openings through which the spinal cord and nerves must pass, and decreasing pressure on the spinal cord and/or nerve roots. The tool can be inserted laterally or posteriorly.

### CROSS-REFERENCE TO RELATED APPLICATIONS

This application is related to U.S. application Ser. No. 11/053,346, entitled INTER-CERVICAL FACET IMPLANT AND METHOD, filed Feb. 8, 2005 [Our Reference No. SFMT-0122US1]; U.S. application Ser. No. 11/053,399, entitled INTER-CERVICAL FACET IMPLANT AND METHOD, filed Feb. 8, 2005 [Our Reference No. SFMT-01118US1]; U.S. application Ser. No. 11/053,624, entitled INTER-CERVICAL FACET IMPLANT AND METHOD, filed Feb. 8, 2005 [Our Reference No. SFMT-01118US2]; U.S. application Ser. No. 11/053,735, entitled INTER-CERVICAL FACET IMPLANT AND METHOD, filed Feb. 8, 2005 [Our Reference No. SFMT-0118US3, which are each, expressly incorporated herein in full, by reference.

### TECHNICAL FIELD

This invention relates to a facet joint implant sizing tool used for sizing implants prior to insertion between the spinous processes.

### BACKGROUND OF THE INVENTION

The spinal column is a bio-mechanical structure composed primarily of ligaments, muscles, vertebrae and intervertebral disks. The bio-mechanical functions of the spine include: (1) support of the body, which involves the transfer of the weight and the bending movements of the head, trunk and arms to the pelvis and legs, (2) complex physiological motion between these parts, and (3) protection of the spinal cord and the nerve roots.

As the present society ages, it is anticipated that there will be an increase in adverse spinal conditions which are characteristic of older people. By way of example only, with aging comes an increase in spinal stenosis (including, but not limited to, central canal and lateral stenosis), and facet arthropathy. Spinal stenosis results in a reduction foraminal area (i.e., the available space for the passage of nerves and blood vessels), which compresses the cervical nerve roots and causes radicular pain. Humpreys, S. C. et al., Flexion and traction effect on C5-C6 foraminal space, Arch. Phys. Med. Rehabil., vol. 79 at 1105 (September 1998). Another symptom of spinal stenosis is myelopathy, which results in neck pain and muscle weakness. Id. Extension and ipsilateral rotation of the neck further reduces the foraminal area and contributes to pain, nerve root compression, and neural injury. Id.; Yoo, J. U. et al., Effect of cervical spine motion on the neuroforaminal dimensions of human cervical spine, Spine, vol. 17 at 1131 (Nov. 10, 1992). In contrast, neck flexion increases the foraminal area. Humpreys, S. C. et al., supra, at 1105.

In particular, cervical radiculopathy secondary to disc herniation and cervical spondylotic foraminal stenosis typically affects patients in their fourth and fifth decade, and has an annual incidence rate of 83.2 per 100,000 people (based on 1994 information). Cervical radiculopathy is typically treated surgically with either an anterior cervical discectomy and fusion (“ACDF”) or posterior laminoforaminotomy (“PLD”), with or without facetectomy. ACDF is the most commonly performed surgical procedure for cervical radiculopathy, as it has been shown to increase significantly the foramina dimensions when compared to a PLF.

It is desirable to eliminate the need for major surgery for all individuals, and in particular, for the elderly. Accordingly, a need exists to develop spine implants and associated instruments that help facilitate insertion of implants to alleviate pain caused by spinal stenosis and other such conditions caused by damage to, or degeneration of, the cervical spine.

### BRIEF DESCRIPTION OF THE DRAWINGS

This invention is described with respect to specific embodiments thereof. Additional aspects can be appreciated from the Figures in which:

FIG. 1 shows a lateral view of two adjacent cervical vertebrae and spinous processes, highlighting the cervical facet joint;

FIG. 2 depicts a lateral view of the cervical spine with spinal stenosis;

FIG. 3A depicts correction of cervical stenosis or other ailment with a wedge-shaped implant positioned in the cervical facet joint;

FIG. 3B depicts correction of cervical kyphosis or loss of lordosis with a wedge-shaped implant with the wedge positioned in the opposite direction as that depicted in FIG. 3A;

FIG. 4A shows correction of cervical stenosis or other ailment with an implant, comprising a facet implant with cleats;

FIG. 4B depicts correction of cervical stenosis or other ailment with an implant, comprising a facet implant with screw fixation;

FIG. 5A depicts a perspective view of a facet implant with screw fixation;

FIG. 5B depicts a perspective exploded view of the implant shown in FIG. 5A;

FIG. 6 shows a perspective view of an implant;

FIG. 7 depicts a posterior view of an implant shown in FIG. 6;

FIG. 8A depicts a perspective view of an embodiment of the invention where the tool head has substantially the dimensions of an implant (head thickness 3 mm) and is pivotably linked to the handle of the tool;

FIG. 8B shows a perspective side view of an embodiment of the invention where cleats are present on the surface of the head and are adapted to contact the facet joint;

FIG. 8C shows an anterior view of an embodiment of the invention where the head is pivoted at about 45 degrees to the axis of the handle and where cleats are present on the surface;

FIG. 8D shows a top view of an embodiment of the invention where the head is oriented parallel with the handle;

FIG. 9A shows a perspective side view of a further embodiment of the invention shown in FIG. 8A, where the tool head thickness dimension is increased to 4 mm;

FIG. 9B shows a perspective side view of a further embodiment of the invention shown in FIG. 9A, where the tool head thickness dimension is increased to 5 mm;

FIG. 10A depicts an enlarged side view of a further embodiment of the invention, where the tool head is about parallel with the base and about 45 degrees to the axis of the handle;

FIG. 10B depicts an enlarged side view of a further embodiment of the invention, where the tool head is about parallel with the axis of the handle;

FIG. 11 depicts an enlarged side view of a further embodiment of the invention, where the tool head is about parallel with the base;

FIG. 12A is a side view of still another embodiment of an implant in accordance with the present invention;

FIG. 12B is a top view of the implant of FIG. 12A;

FIG. 12C is a bottom view of the implant of FIG. 12A;

FIG. 12D is a side view of the implant of FIG. 12A illustrating the various arrangements of a bone screw associated the implant;

FIG. 13 is a flow chart illustrating the steps involved in the use of an embodiment of the invention; and

FIG. 14 is a flow chart illustrating an alternative embodiment of a method in accordance with the present invention.

### DETAILED DESCRIPTION

Embodiments of the present invention provide for a facet joint implant sizing tool used for sizing implants prior to insertion of an implant between the facet joint. By guiding the selection of a correctly sized implant, the tool facilitates insertion of minimally invasive surgical implants that preserves the physiology of the spine. In embodiments of the invention, the facet joint implant sizing tool provides for sizing an implant in order to distract the cervical spine to for example increase the foramina dimension in extension and neutral positions. Such implants distract, or increase the space between, the vertebrae to increase the foraminal area or dimension, and reduce pressure on the nerves and blood vessels of the cervical spine.

In an embodiment of the invention, a joint and base of a sizing tool closely resemble the overall dimensions and shape of the implant in order to best determine the appropriate dimension of an implant. In an embodiment of the invention, the sizing tool contains cleats on the tool joint surface wherein the cleats embed the sizing tool in the facet joint.

In an embodiment of the invention, the sizing tool facet joint hereinafter ‘joint’ pivots and rotates about a base. In an embodiment of the invention, the sizing tool contains a bore which replicates the approximate location of the bore in the implant used for affixing the implant to the lateral mass In an embodiment of the invention, the sizing tool contains a bore, which replicates the location of the bore in the implant and is used for punching or drilling a pivoting bore in the lateral mass to assist in inserting the implant.

In various embodiments of the invention, the sizing tool has a joint dimension varying from 1.5 mm to 5 mm in width. In other embodiments of the invention, other interfacet spacer dimensions can also be varied in the facet joint implant sizing tool in order to select the most appropriate implant. The present embodiments of the invention also preserve mobility of the facet joints during sizing with the facet joint implant sizing tool.

Further embodiments of the present invention accommodate the distinct anatomical structures of the spine, minimize further trauma to the spine, and obviate the need for invasive methods of surgical implantation. Embodiments of the present invention also address spinal conditions that are exacerbated by spinal extension.

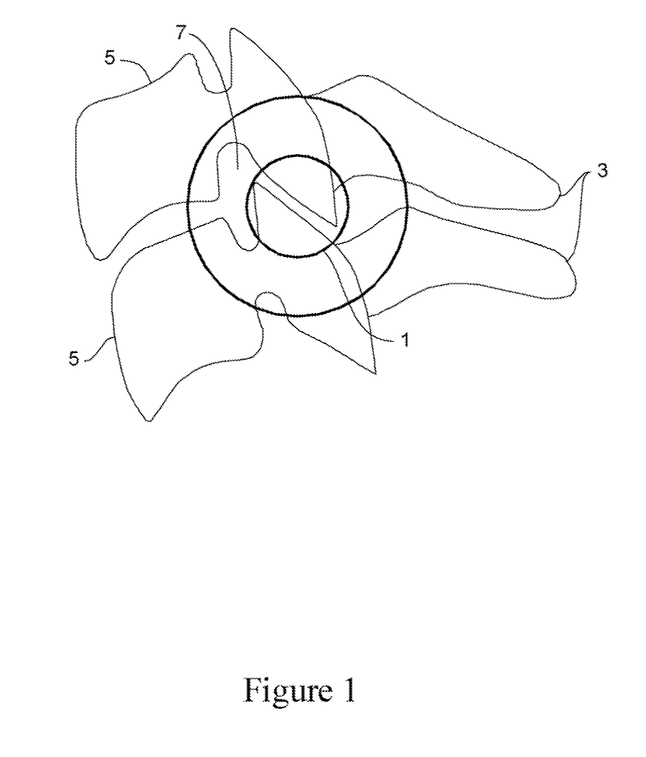

FIG. 1 shows a simplified diagram of a portion of the cervical spine, focusing on a cervical facet joint 1 formed between two adjacent cervical vertebrae. The spinous processes 3 are located posteriorly and the vertebral bodies 5 are located anteriorly, and a nerve root canal 7 is visible.

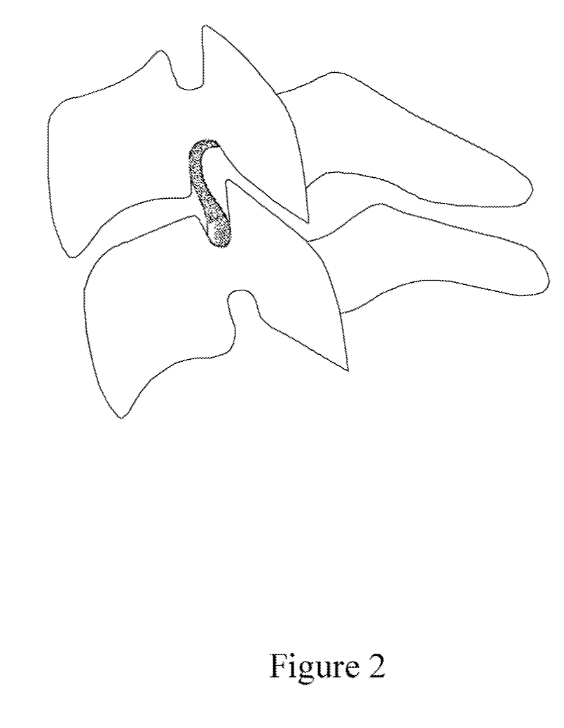

FIG. 2 depicts cervical foraminal stenosis. From the drawing, the nerve root canal 7 is narrowed relative to the nerve root canal 7 depicted in FIG. 1. The spinal canal and/or intervertebral foramina also can be narrowed by stenosis. The narrowing can cause compression of the spinal cord and nerve roots.

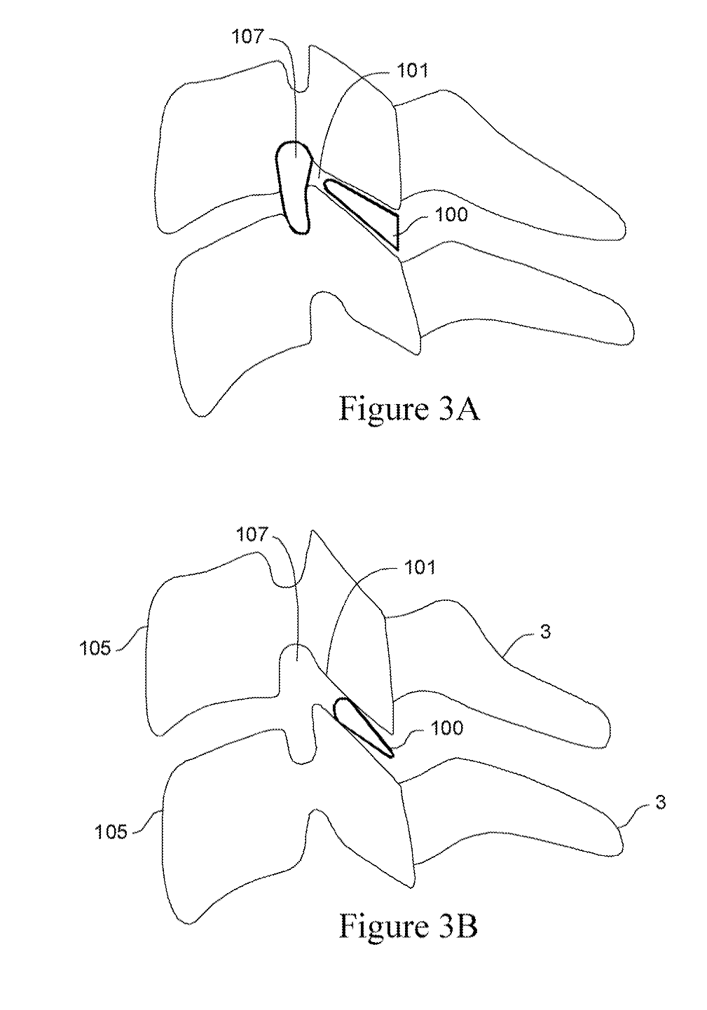

FIG. 3A shows an embodiment 100 of an implant, which distracts at least one facet joint, in order to increase the dimension of the neural foramen while retaining facet joint mobility. In embodiment 100 the implant is wedge-shaped and can be positioned in the cervical facet joint 101 to distract the joint and reverse narrowing of the nerve root canal 107. In this embodiment 100, the implant is positioned with the narrow portion of the wedge facing anteriorly. In another embodiment 100 (FIG. 3B), the wide portion of the wedge faces anteriorly, to correct for cervical kyphosis or loss of cervical lordosis.

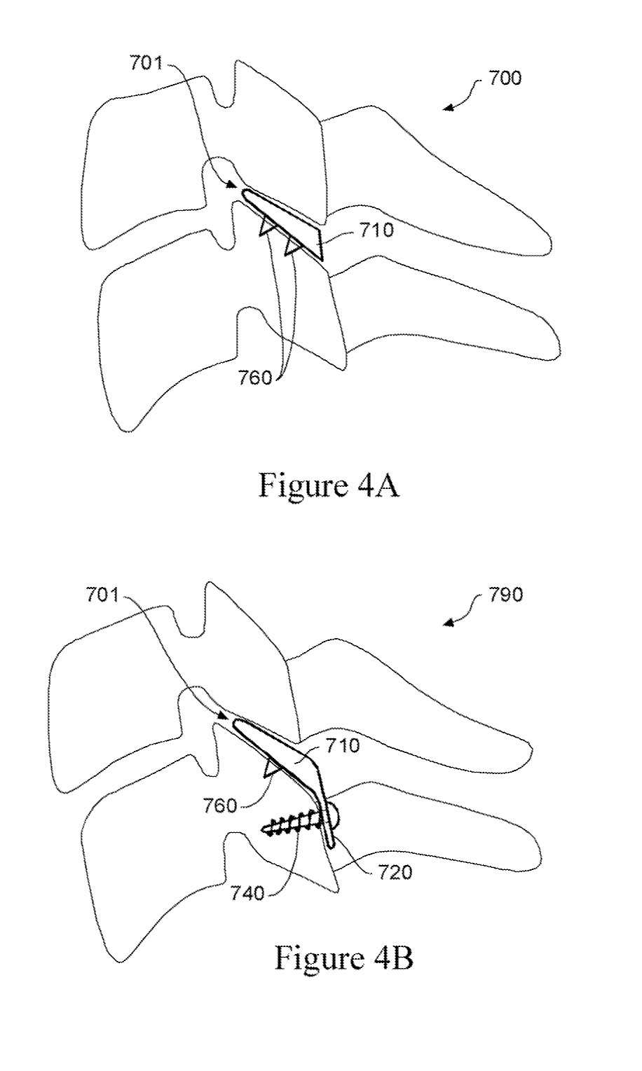

A further alternative embodiment 700 of an implant, is illustrated in FIG. 4A. In this embodiment 700, the joint implant 710 has on a lower side at least two cleats 760. In other embodiments, a plurality of cleats 760 is preferable. The cleats 760 are able to embed in the bone of the cervical facet joint 701 to facilitate retention of the implant 700 in the joint 701. The cleats 760 can face in a direction substantially opposite the direction of insertion, for retention of the implant 700. In various embodiments, the joint implant 710 can be wedge-shaped or substantially even in thickness, depending upon the desired distraction. An alternative embodiment 800 of an implant, is illustrated in FIG. 4B. In this embodiment 790, the joint insert 710 has on a lower side at least one tooth 760 and a screw 740 to affix the insert plate 720 to the facet joint. The cleats 760 are able to embed in the bone of the cervical facet joint 701 to facilitate retention of the implant.

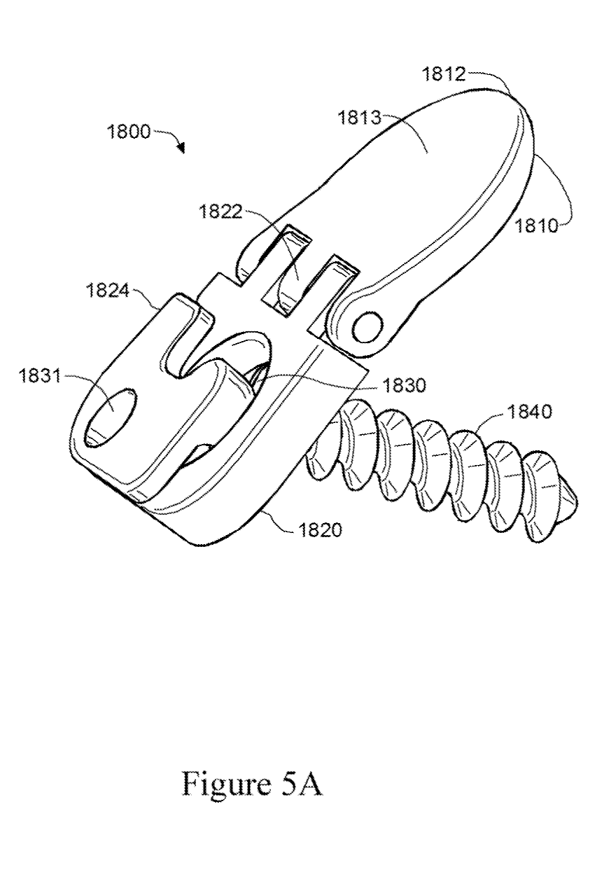

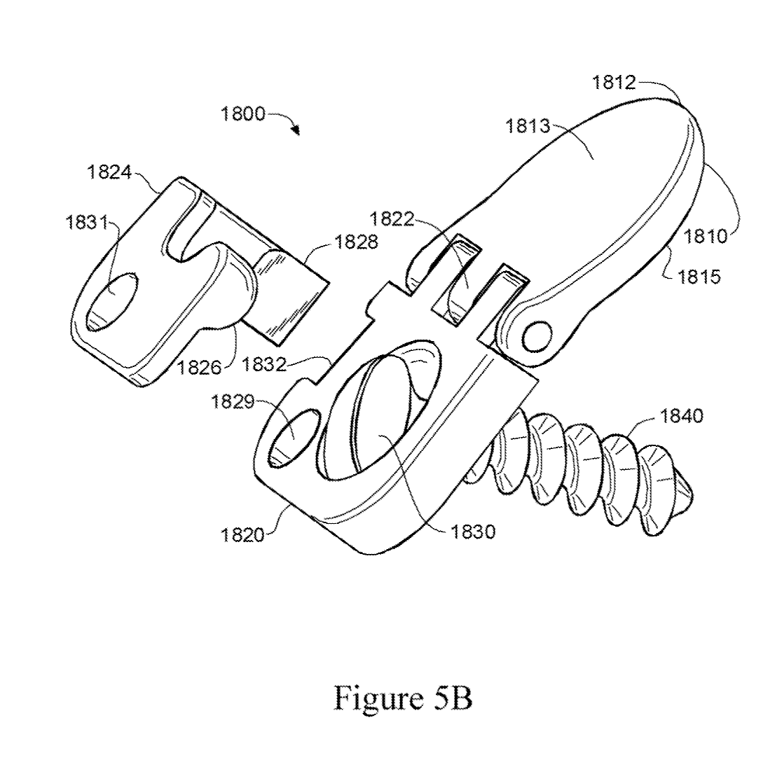

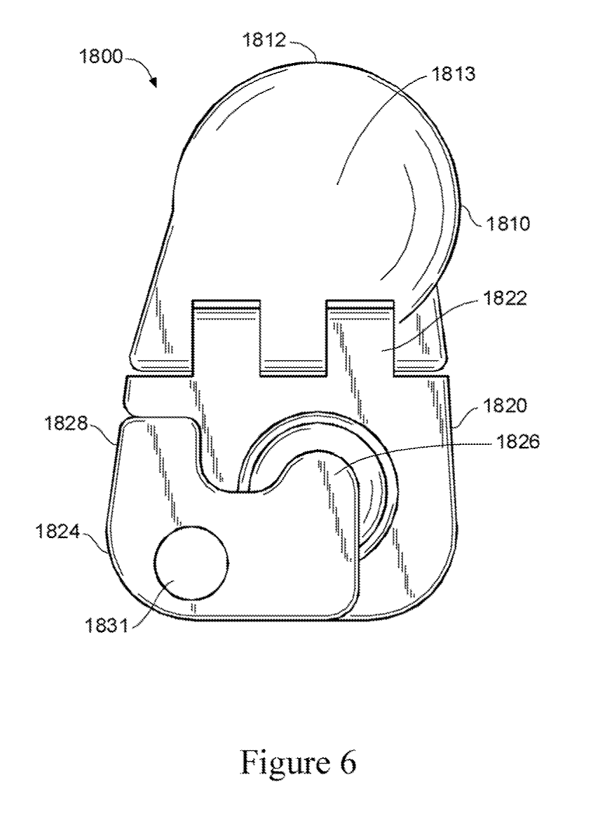

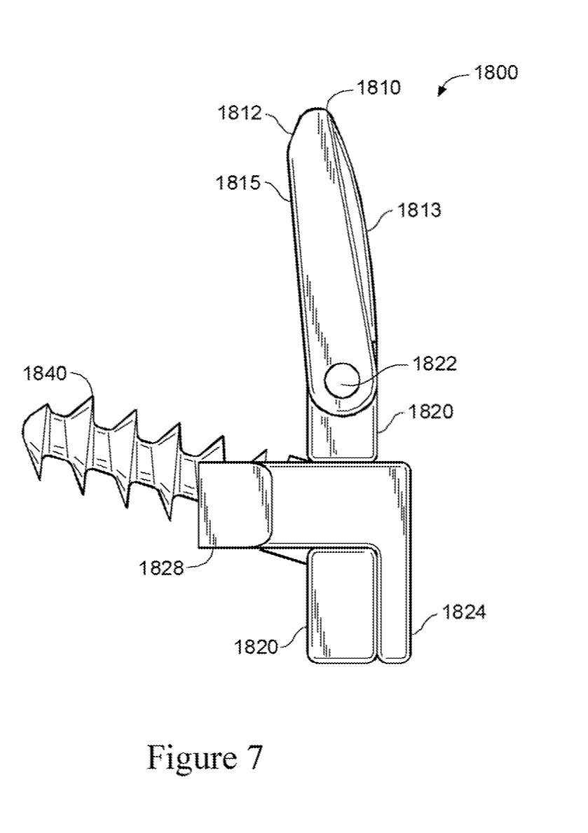

FIG. 5A and FIG. 5B depict a further embodiment 1800 of the implant. In this embodiment, an artificial facet joint spacer 1810 is connected with a lateral mass plate 1820 with a pivot 1822. The pivot 1822 allows the lateral mass plate 1820 to bend at a wide range of angles relative to the artificial facet joint and preferably at an angle of more than 90 degrees, and this flexibility facilitates positioning and insertion of the artificial facet joint spacer 1810 into a patient's facet joint, the anatomy of which can be highly variable among individuals. The pivot 1822 further facilitates customizing the anchoring of the implant, i.e., the positioning of a fixation device. The pivot enables positioning of the lateral mass plate 1820 to conform to a patient's cervical spinal anatomy, and the lateral mass plate 1820 accepts a fixation device to penetrate the bone. In an embodiment of the present invention, the spacer can be made of bone. The artificial facet joint spacer 1810 can be curved or rounded at a distal end 1812 (FIG. 6), and convex or dome-shaped on a superior surface 1813 to approximate the shape of the bone inside the facet joint. The inferior surface 1815 can be flat or planar (FIG. 7).

The lateral mass plate 1820, when implanted in the spine, is positioned outside the facet joint, preferably against the lateral mass or against the lamina. The lateral mass plate 1820 has a bore 1830 there through. The bore 1830 can accept a bone screw 1840, also referred to as a lateral mass screw, to secure the lateral mass plate 1820 preferably to the lateral mass or alternatively to another part of the spine, and thus to anchor the implant. The lateral mass screw 1840 preferably has a hexagonal head to accept an appropriately-shaped wrench.

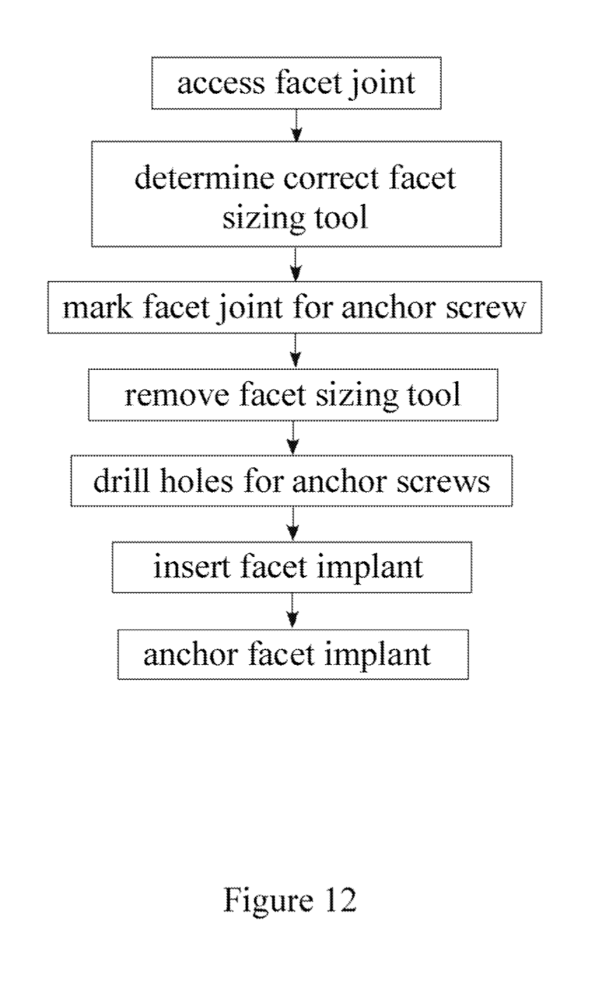

A further embodiment of an implant 2600 in accordance with the present invention is shown in FIGS. 12A-12D. The implant 2600 resembles implants in that the facet joint spacer 2610 has limited freedom of movement relative to the lateral mass plate 2620. As can be seen, a hinge 2622 connects the facet joint spacer 2610 with the lateral mass plate 2620, allowing the facet joint spacer to pivot up and down relative to a plane of the lateral mass plate 2620. However, in other embodiments the facet joint spacer 2610 can be connected with the lateral mass plate 2620 by way of a spheroidal joint arrangement or by way of some other structure.

An inferior surface 2615 of the facet joint spacer 2610 includes a plurality of cleats 2686 extending from the inferior surface 2615. In one example as seen in FIG. 12A the cleats point in a direction that is opposed to the direction of insertion of the facet joint spacer in the facet joint in order to ease the insertion step and to aid in preventing the facet joint spacer from backing out of the facet joint. Additionally the cleats have, in one embodiment, a thickness that is less that the thickness of the facet joint spacer defined between a superior surface of the facet joint spacer and an inferior surface vertebra of a facet joint spacer. The plurality of cleats 2686 can penetrate or grip a superior facet of a lower vertebra of the targeted facet joint, thereby reducing slippage of the facet joint spacer 2610 relative to the superior facet. The cleats 2686 do not directly restrict the inferior facet of an upper vertebra of the targeted facet joint from moving along the superior surface 2613 of the facet joint spacer 2610. The cleats 2686 can further promote bone growth by roughing the surface, which can provide beneficial results where an increase in surface contact resulting in a reduction of slippage is desired. In a preferred embodiment the facet joint spacer 2610 can include a inferior surface 2615 connected with the hinge 2622 and formed of a light-weight, bio-compatible material having a desired strength, such as titanium, titanium alloys, aluminum, aluminum alloys, medical grade stainless steel, etc. Such a structure is also referred to herein as an inferior shim 2680. As shown, a substantial portion of the facet joint spacer 2610 including the superior surface 2613 can be formed of a biocompatible polymer, such as described below. Such a substantial portion is also referred to herein as a superior shim 2682. Such a material is radiolucent, and can have a desired smoothness and reduced compressive strength relative to the inferior surface 2615 such that the superior surface 2613 of the facet joint spacer 2610 allows for a desired slippage relative to the inferior facet of the facet joint. A superior surface 2613 having a reduced compressive strength and an increased elasticity relative to a bony structure of the spine. The superior shim 2682 can be molded onto the inferior shim 2680 to form the facet joint spacer 2610, or the superior shim 2682 can be adhesively fastened to the inferior shim 2680, interference fit with optional protuberances of the inferior shim 2680, etc. One of ordinary skill in the art will appreciate the different techniques for fixedly connecting a superior shim 2682 with the inferior shim 2680. The lateral mass plate 2620 can be made of any of the materials described herein. Preferably the lateral mass plate 2620 can be comprised of a biocompatible polymer as described herein.

It is also to be understood that the inferior shim can be comprised of a rigid material while the superior shim can be comprised of a more compliant and/or compressible material. Thus the inferior shim can carry the load experienced in the facet joint while the superior shim can be more compliant. The facet joint spacer can, for example, be comprised of one material that has been formed to have a gradient of stiffness from more stiff in the area of the inferior shim to less stiff and more compliant in the area of the superior shim. For example a PEEK polymer material as described below can be formed in the area of the inferior shim with fillers that increase the stiffness and strength of the material while the PEEK polymer in the area of the superior shim does not have such fillers and is thus more compliant.

In a preferred embodiment, the cleats 2686 of the implant 2600 can extend from the inferior surface 2615 to have a saw-tooth shape and arrangement to resist movement in a generally posterior direction away from the facet joint (i.e., toward the lateral mass plate 2620 as shown) and further to resist movement in a lateral direction relative to the facet joint. However, the cleats 2686 need not necessarily be saw-tooth in shape and arrangement. For example, the cleats 2686 can have a conical shape, a pyramid shape, a curved shape, etc. Further, as shown particularly in FIG. 12C four cleats 2686 extend from the inferior surface 2615. In other embodiments, any number of cleats 2686 can be provided, the cleats 2686 being similarly sized and shaped, or varying in size and shape. In reflection on the teachings contained herein, one of ordinary skill in the art will appreciate the myriad different shapes with which the cleats 2686 can be formed. The cleats 2686 can vary in performance and technique for implantation with shape and number; however, the present invention is meant to encompass all such variations.

The implant 2600 can further optionally include plate cleats 2688 extending from a surface of the lateral mass plate 2620 substantially contacting the bony structures of the spine (e.g., the lateral mass). The plate cleats 2688 can help anchor the lateral mass plate 2620 in position either to assist in resisting shifting as a bone screw 2640 is associated with the bony structure, or as an adjunct to the bone screw 2640. Surface roughening caused by the plate cleats 2688 can further promote bone growth near and/or integrally with the lateral mass plate 2620. As shown particularly in FIG. 38C there are four plate cleats 2688, each plate cleat 2688 having a conical structure. However, as above the plate cleats 2688 can vary in size, number and shape. For example, the plate cleats 2688 can have a saw-tooth shape, a pyramid shape, a curved shape, etc.

Referring to FIG. 12D, a bone screw 2640 of the implant 2600 can be arranged in a bore 2630 so that the bone screw 2640 and bore 2630 permit a relative degree of freedom of movement resembling a ball-in-socket joint. Such an arrangement can allow for flexibility in fastening the implant 2600 to a bony structure, thereby allowing a surgeon to avoid diseased or fragile bony structures, fastening the implant 2600 to more durable, healthy bony structures. The bone screw 2640 can swivel within the bore 2630 toward or away from the facet joint spacer 2610 and/or from side-to-side relative to the facet joint spacer 2610. When the bone screw 2640 is arranged as desired a retaining plate 2624 (FIG. 12B) can be attached to the lateral mass plate 2620 to resist backing out of the bone screw 2640, similar to the functioning of features as shown in previous embodiments. As can be seen in FIG. 12B, retaining plates 2624 can have a projection 2625 that fits in a recess 2627 of the lateral mass plate 2620 in order to prevent rotation of the retaining plate 2624 once screw 2640 is tightened against retaining plate 2624.

As described above in reference to FIGS. 12A-D, the cleats 2686 are saw-tooth in shape and arrangement, but alternatively can have some other shape and/or arrangement. For example, the cleats 2686 can have a pyramidal shape, a curved shape, a conical shape, etc. Further, the shape, size and arrangement for cleats 2686 of the inferior surface 2615 can be different or the same from cleats 2686 of the superior surface 2613. The shape, size, and arrangement of the cleats 2686 can be chosen based on the location of implantation, the preferences of the surgeon, the physical condition of the target facet joint, etc. In an embodiment of the present invention, the spacer can be made of bone.

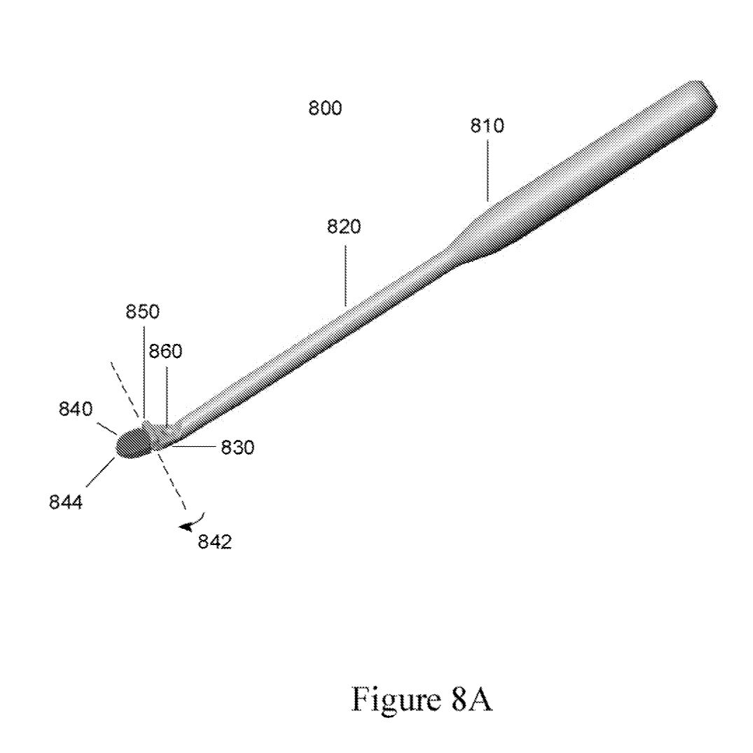

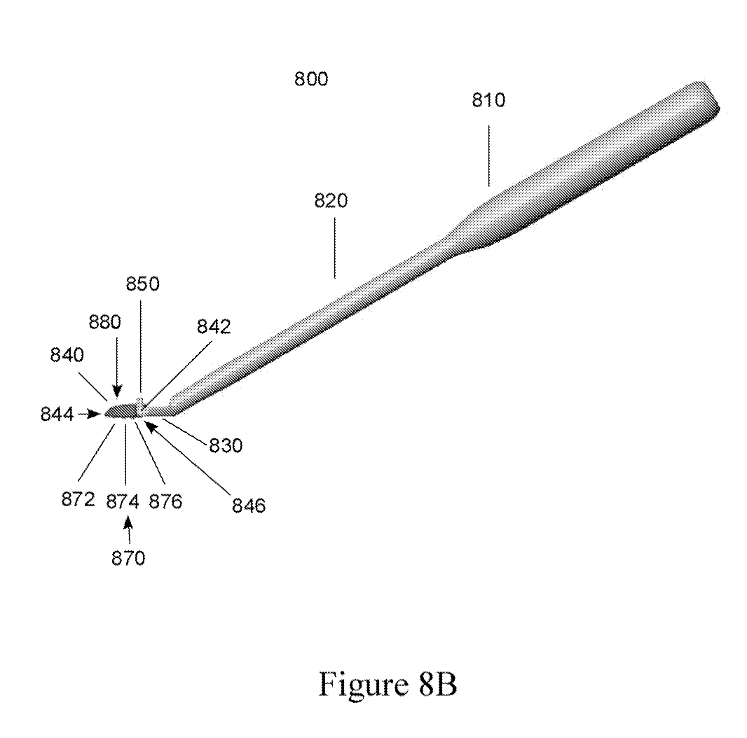

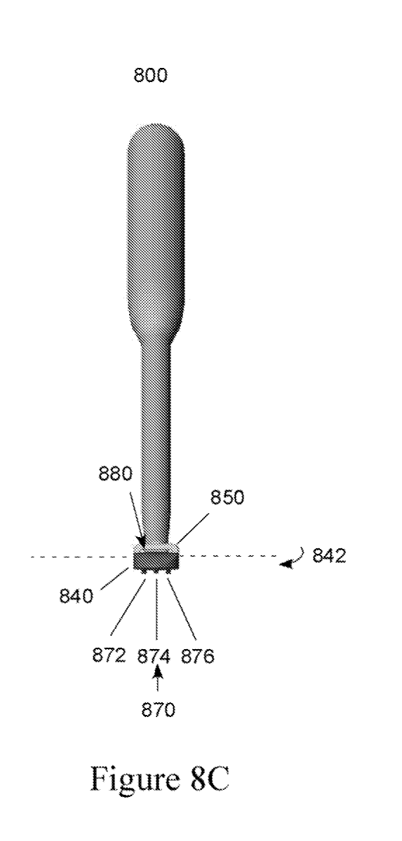

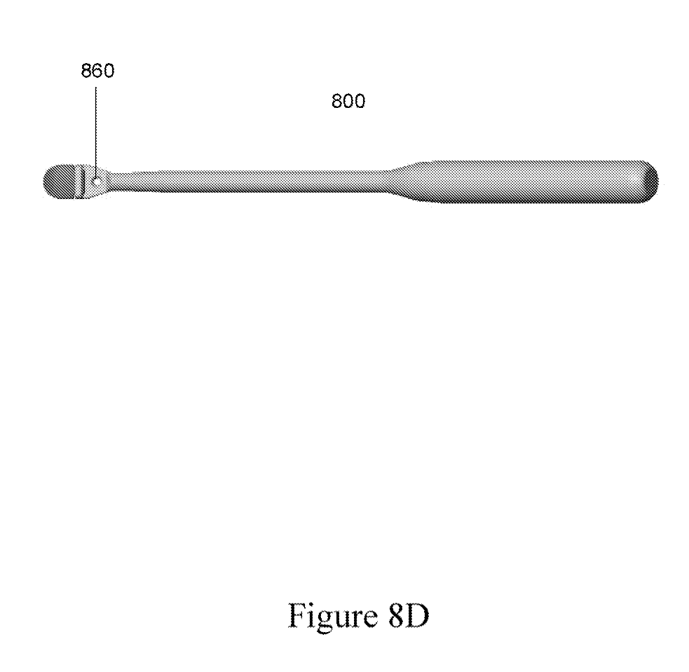

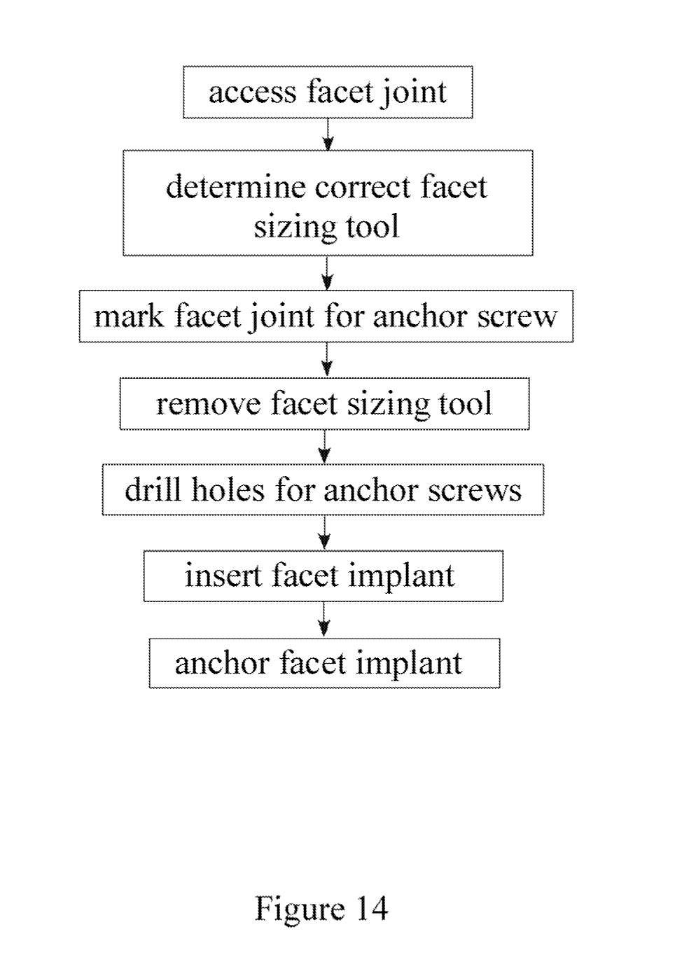

FIG. 14 is a flow chart of an embodiment of a method in accordance with the present invention for implanting an implant as described in FIGS. 12A through 12D. An incision must first be made to expose the surgical site and access the targeted facet joint (step 1410). Once the facet joint is made accessible, the facet joint can be sized and distracted (step 1420). A sizing tool 800 (for example, see FIG. 8) can be inserted to select the appropriate size of an implant 2600 of the invention for positioning in the cervical facet joint. This step may be repeated as necessary with, if desired, different sizes of the tool 800 until the appropriate size is determined. This sizing step also distracts the facet joint and surrounding tissue in order to facilitate insertion of the implant 2600. It is to be understood that the final position of the lateral mass plate 2620 relative to the facet joint spacer 2610 will depend on the actual spine configuration. Once the lateral mass plate 2620 is positioned, or prior to the positioning of the lateral mass plate 2620, a bore can be drilled in the bone to accommodate the bone screw 2640 (steps 1430-1450). Alternatively the screw 2540 can be self-tapping. The screw 2540 is then placed through the first bore 2530 and secured to the bone (step 1470), preferably the lateral mass or the lamina, thereby holding the facet joint spacer 2610 in place. In order to lock the bone screw 2540 in place and to lock the position of the facet joint spacer 2610 and the lateral mass plate 2620 in place, a self-tapping locking screw 2690 is positioned within a second bore of the lateral mass plate 2620 and secured to the bone, thereby resisting undesirable movement of the lateral mass plate 2620 (step 1480). A head of the locking screw 2690 can further block movement of the bone screw 2640 by trapping the bone screw head between the locking screw head and the first bore. The locking screw 2690 therefore prevents the lateral mass plate 2620 and the facet joint spacer 2610 from rotating and, as previously indicated, prevents the bone screw 2640 from backing out from the vertebra.

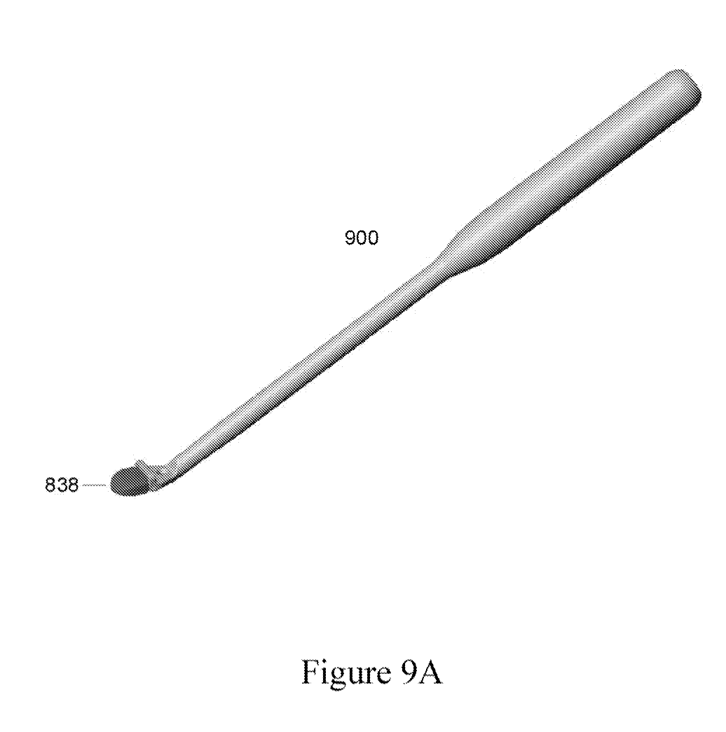

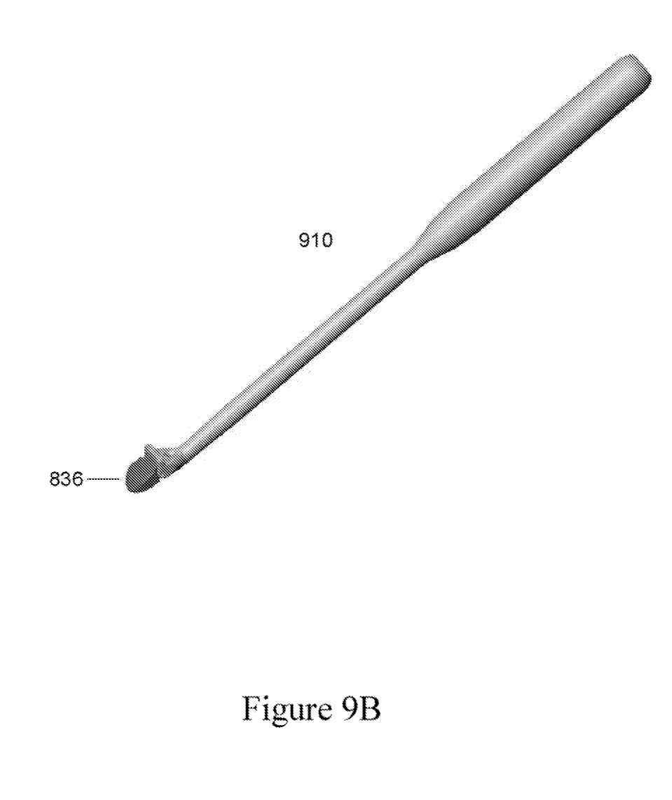

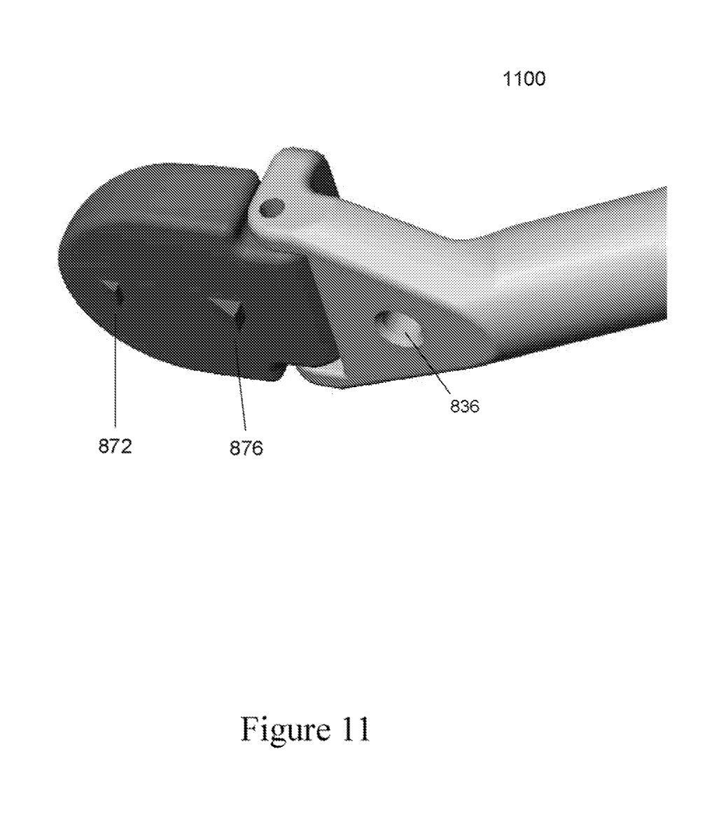

In embodiments of the present invention, a facet joint implant sizing tool is used for sizing implants prior to insertion of the implant between the spinous processes. FIG. 8A depicts a facet joint implant sizing tool 800, which is designed to be inserted between one facet joint, in order to measure the dimension of an implant. The implant can for example be used to increase the dimension of the neural foramen while retaining facet joint mobility. The tool has a handle 810, a beam 820, and a base 830, upon which a joint sizer 840 can pivot and rotate about axis 842. The joint sizer 840 is shaped and dimensioned corresponding to the shape and dimension of an artificial facet joint spacer (e.g., 1810 in FIG. 5). In various embodiments of the invention, the joint sizer 840 can have essentially the same shape, dimensions and other features as the artificial facet joint spacer 1810, with the exception that the dimensions of the joint sizer 840 will vary, in order to be able to use different sizing tools 836, 838, 840 (FIGS. 8A, 9A and 9B) to determine the dimensions of the cervical facet joint and select an appropriately-sized implant. The joint spacer 840 preferably can be used to distract the facet joint prior to the step of implanting the implant in the facet joint. In this regard, the joint spacer 840 is rounded or tapered at the most distal point 844, to facilitate insertion into a cervical facet joint, see also the tool 1100, shown in FIG. 11. The joint spacer 840 also can have a slightly convex superior surface, the degree of convexity varying among different sizing tools in order to determine the desired degree of convexity of an implant to be implanted in the cervical facet joint. The joint spacer 840 may have a uniform thickness along a proximal mid-section 846. Accordingly, the inferior surface can be concave. Alternatively, the proximal mid-section may be convex on the superior surface without being uniform in thickness. Thus, the inferior surface can be flat or planar. The joint spacer 840 also can be curved. The curved shape of the joint spacer 840 can fit the shape of a cervical facet joint, which is comprised of an inferior facet of an upper vertebra and a superior facet of a lower adjacent vertebra. The convex shape of the superior surface of the artificial facet joint fits with a concave shape of the inferior facet of the upper cervical vertebrae. The concave shape of the inferior surface of the joint spacer 840 fits with the convex shape of the superior facet of the cervical vertebrae. The degree of convexity and concavity of the joint spacer 840 inferior and superior surfaces can be varied to fit a patient's anatomy and the particular pairing of adjacent cervical vertebrae to be treated. In various embodiments, the joint spacer 840 can be wedge-shaped or substantially even in thickness, depending upon the desired distraction.

The facet joint implant sizing tool 800 has a stop 850 to limit the insertion of the joint spacer 840 of the sizing tool 800 into the facet joint. At the distal end of the base 830, upon which the proximal end of the joint spacer 840 can pivot, the stop 850, or protuberance is also present to restrict the angle that the joint spacer 840 can be rotated relative to the beam 820. The stop 850 can be a ridge that separates the joint spacer 840 from the base 830. Alternatively, the stop 850 can be any structure that prevents insertion beyond the stop 850, including pegs, teeth, and the like. As shown in FIGS. 8B and 8C, the rotation of the proximal end 846 of the joint spacer 840 in a clockwise orientation about the axis 842 is restricted by the stop 850. In a further alternative embodiment the cleats 872, 874, 876 (FIG. 11) can face in a direction substantially opposite the direction of insertion, for retention of the tool 800. Other embodiments of the present invention are envisaged which are otherwise enabled to bend by some equivalent structure or material property.

In embodiments of the invention, a joint spacer 840 and a base 830 of a sizing tool closely resemble the dimension and shape of an implant which can be inserted between the facet joint, in order to best determine the appropriate dimension of implants. FIG. 8B shows an embodiment 800 of a facet joint implant sizing tool which contains cleats 872, 874, 876 (FIG. 11) on the surface 870 of the joint spacer 840 which engage the superior articular processes of one vertebrae while the surface engaging the inferior articular process of the above vertebrae, is smooth in order to facilitate sliding the joint spacer 840 into the gap between these processes. In an embodiment of the invention, the cleats 872, 874, 876 can be spaced over the length of the surface 870 of the joint spacer 840. As shown in FIG. 8C in an embodiment of the invention, the cleats 872, 874, 876 can be spaced over the breadth of the surface 870 of the joint spacer 840. This is similar in design to the artificial joint spacer depicted in FIG. 12C. In an embodiment of the invention, a joint spacer 840 of a sizing tool 800 can be similar to the artificial facet joint spacer 1810 of the implant 1800, (see FIG. 5), while a base 830 of a sizing tool 800 can be similar to the lateral mass plate 1820 of the implant 1800 in order to best determine the appropriate dimension of an implant. When the tool is held by a handle 810, a joint spacer 840 can be aligned with a base 830 and inserted between the articular processes, where one or more cleats 872, 874, 876 initially slide past the surface of the articular processes, but eventually become embedded in the superior articular process as pressure is exerted on the joint spacer 840 from the other side 880 of the joint spacer 840 pressing against the above inferior articular process.

The pivot 842 allows the joint spacer 840 to bend at a wide range of angles relative to the base 830 and preferably at an angle of up to and more than 90 degrees and this flexibility facilitates positioning and insertion of the tool 800 into a patient's facet joint, the anatomy of which can be highly variable among individuals. The pivot 842 further facilitates customizing the anchoring of the tool. The pivot enables positioning of the base 830 and joint spacer 840 to conform to a patient's cervical spinal anatomy. The joint spacer 840 can be curved or rounded at a distal end, and convex or dome-shaped on a superior surface 880 to approximate the shape of the bone inside the facet joint. The inferior surface 870 can be flat or planar. Alternatively, the inferior surface 870 can be concave. In another alternative embodiment of the invention, the inferior surface 870 can be convex.

In an embodiment of the invention, the joint spacer 840 is positioned with the narrow portion of the wedge facing anteriorly. In another embodiment of the invention, the wide portion of the wedge faces anteriorly, to correct for cervical kyphosis or loss of cervical lordosis.

FIG. 8D shows an embodiment 800 of a facet joint implant sizing tool which contains a bore 860 which replicates the approximate location of the bore in the implant used for affixing the implant to the lateral mass. In various embodiments of the invention, the base 830, when positioned rests against the lateral mass or against the lamina. The bore 860 can accept a bone screw, to temporarily secure the base 830 preferably to the lateral mass or alternatively to another part of the spine. The bore 860 allows the appropriate location of the retention bore of the implant to be determined. In an embodiment of the invention 800, the sizing tool contains a bore 860 which replicates the location of the bore in the implant and is used for punching or drilling a locating bore in the lateral mass to assist in inserting the implant.

Different sizing tools 800 covering a range of dimensions of the joint spacer 836, 838, 840 can be inserted successively into a cervical facet joint to select the appropriate size of an implant to position in the cervical spine, with the appropriate convexity and concavity of artificial facet joint. Each preferably larger head also can be used to distract the facet joint. In various embodiments of the invention, the facet joint implant sizing tool has a joint dimension varying from about 1.5 mm to about 5 mm or more in width to increase foramina dimension in extension and neutral. As shown in FIGS. 9A and 9B, the joint 838, 836 dimension of the tools 900, 910 is increased from 3 mm (see FIG. 8A) to 4 mm 838 and 5 mm 836 respectively. In other embodiments of the invention, other interfacet spacer dimensions can also be varied in the facet joint implant sizing tool in order to select the most appropriate implant. The present embodiments of the invention allow the tool to be used to size the appropriate implant while otherwise preserving the mobility of the facet joints.

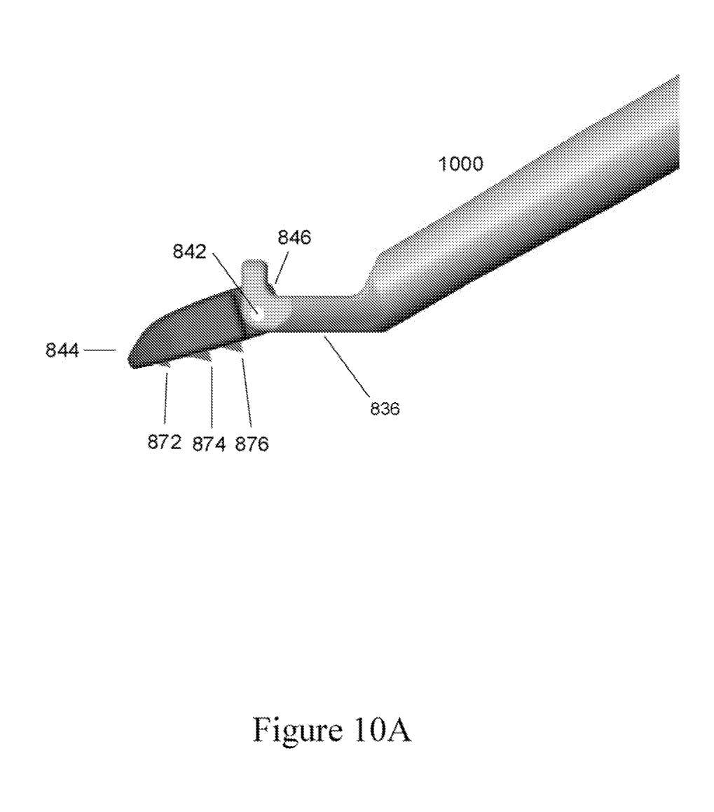

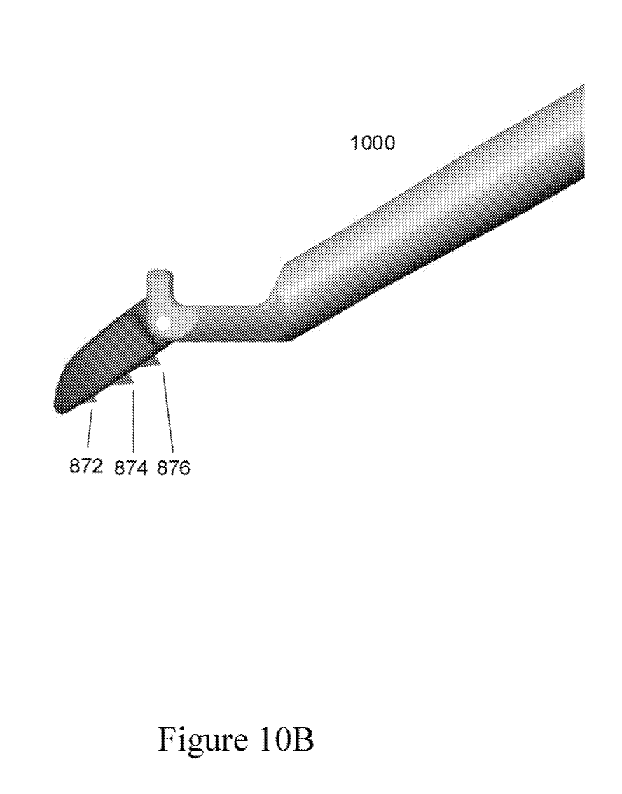

In various embodiments of the invention, the distal end 844 of the joint spacer 840 is tapered in thickness to facilitate insertion of the tool 1000 (see FIG. 10A). The tapered distal end 844 meets and is continuous with a proximate end 846, which, in an embodiment, has a uniform thickness, and is connected flexibly, preferably with a pivot point 842, to the base 830 of the tool 1000 (see FIG. 10B).

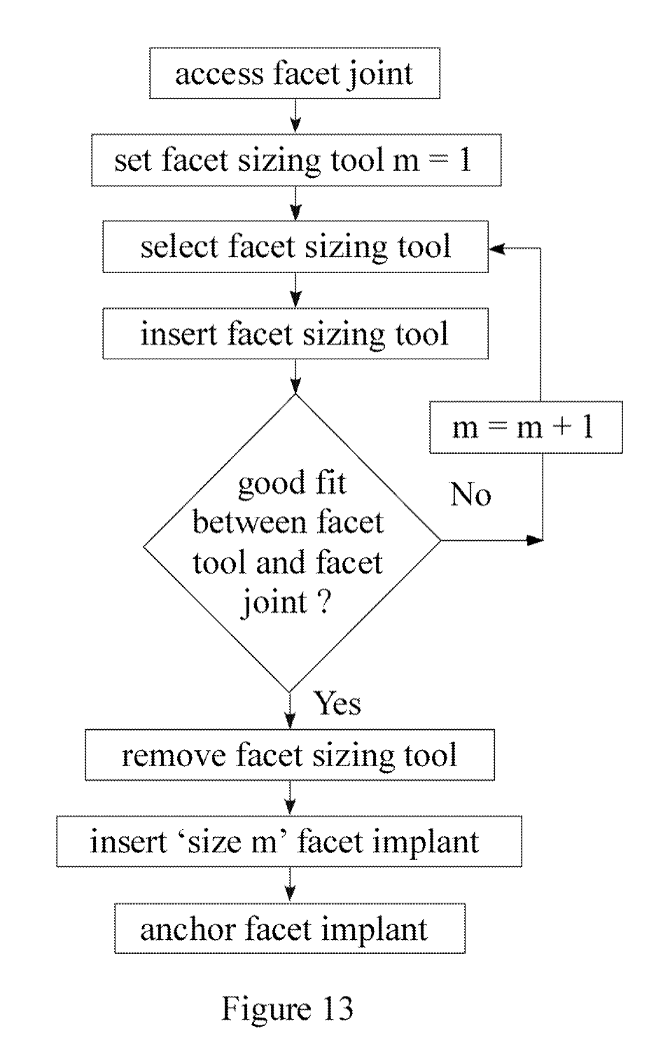

FIG. 13 is a flow chart of the method of use of the facet joint implant sizing tool. An incision must first be made to expose the surgical site and access the targeted facet joint (step 1310). Once the facet joint is made accessible, a facet sizing tool can be selected (steps 1320-1330). A sizing tool 800 (for example, see FIG. 8) can be inserted to select the appropriate size of an implant 2600 of the invention for positioning in the cervical facet joint (step 1340). This inserting step 1340 also distracts the facet joint and surrounding tissue in order to facilitate insertion of the implant 2600. The fit between the sizing tool 800 and the facet joint is evaluated in step 1350. At step 1390 the size of the sizing tool is incremented. Steps 1330, 1340, 1350 and 1390 may be repeated as many times as necessary with increasing size tools 800 until the appropriate size is determined. Once the appropriate size is determine, the physician can select an appropriate facet joint spacer 2610 with the lateral mass plate 2620. The facet joint spacer 2610 can then be urged between the facets into the facet joint (step 1370). It is to be understood that the final position of the lateral mass plate 2620 relative to the facet joint spacer 2610 will depend on the actual spine configuration (step 1380). Preferably the implant is between the C5 and C6 vertebrae level, or the C6 and C7 vertebrae level. It is noted that two implants preferably will be implanted at each level between vertebrae. That is, an implant will be placed in a right facet joint and also in a left facet joint when viewed from a posterior view point. This procedure can be used to increase or distract the foraminal area or dimension of the spine in an extension or in neutral position (without having a deleterious effect on cervical lordosis) and reduce the pressure on the nerves and blood vessels. At the same time this procedure preserves mobility of the facet joint. The facet itself is somewhat shaped like a ball and socket joint. Accordingly, in order to accommodate this shape, the facet joint spacer 2610 can have a rounded leading edge shaped like a wedge or tissue expander to cause distraction of the facet joint as the facet joint spacer is urged into the facet joint of the spine. The facet joint spacer 2610 also includes the convex superior surface 2613 in order to more fully accommodate the shape of the facet joint of the spine. It is possible in the alternative to have a curve-shaped facet joint spacer 2610 with a convex superior surface 2613 and a concave inferior surface 2614, the distal end of the facet joint spacer 2610 tapering to facilitate insertion, while the remainder of the facet joint spacer 2610 has a uniform thickness.

Once the facet joint spacer 2610 is positioned, the lateral mass plate 2620 is tilted and/or swiveled so that the lateral mass plate 2620 is adjacent to the vertebrae and preferably to the lateral mass or to the lamina (step 2512). Thus the lateral mass plate 2620 may be disposed at an angle relative to the facet joint spacer 2610 for a representative spine configuration.

It is to be understood that a facet joint implant sizing tool in accordance with the present invention, and/or portions thereof can be fabricated from somewhat flexible and/or deflectable material. In these embodiments, the facet joint implant sizing tool and/or portions thereof can be made out of a polymer, such as a thermoplastic. For example, in one embodiment, the facet joint implant sizing tool can be made from polyketone, known as polyetheretherketone (“PEEK”). Still more specifically, the facet joint implant sizing tool can be made from PEEK 450G, which is an unfilled PEEK approved for medical implantation available from Victrex of Lancashire, Great Britain. Other sources of this material include Gharda located in Panoli, India. PEEK has the following approximate properties:

| Property | Value |
|---|---|
| Density | 1.3 g/cc |
| Rockwell M | 99 |
| Rockwell R | 126 |
| Tensile Strength | 97 MPa |
| Modulus of Elasticity | 3.5 GPa |
| Flexural Modulus | 4.1 GPa |

The material specified has appropriate physical and mechanical properties and is suitable for carrying and spreading a physical load between the adjacent spinous processes. The facet joint implant sizing tool and/or portions thereof can be formed by extrusion, injection, compression molding and/or machining techniques.

In some embodiments, the facet joint implant sizing tool can comprise, at least in part, titanium or stainless steel, or other suitable implant material which is radiopaque, and at least in part a radiolucent material that does not show up under x-ray or other type of imaging. The physician can have a less obstructed view of the spine under imaging, than with a facet joint implant sizing tool comprising radiopaque materials entirely. However, the facet joint implant sizing tool need not comprise any radiolucent materials.

It should be noted that the material selected also can be filled. For example, other grades of PEEK are also available and contemplated, such as 30% glass-filled or 30% carbon-filled, provided such materials are cleared for use in implantable devices by the FDA, or other regulatory body. Glass-filled PEEK reduces the expansion rate and increases the flexural modulus of PEEK relative to that unfilled PEEK. The resulting product is known to be ideal for improved strength, stiffness, or stability. Carbon-filled PEEK is known to enhance the compressive strength and stiffness of PEEK and to decrease its expansion rate. Carbon-filled PEEK offers wear resistance and load-carrying capability.

In this embodiment 800, the facet joint implant sizing tool is manufactured from PEEK, available from Victrex. As will be appreciated, other suitable similarly biocompatible thermoplastic or thermoplastic polycondensate materials that resist fatigue, have good memory, are flexible, and/or deflectable, have very low moisture absorption, and good wear and/or abrasion resistance, can be used without departing from the scope of the invention. The spacer also can be comprised of polyetherketoneketone (“PEKK”). Other materials that can be used include polyetherketone (“PEK”), polyetherketoneetherketoneketone (“PEKEKK”), and polyetheretherketoneketone (“PEEKK”), and generally a polyaryletheretherketone. Further, other polyketones can be used as well as other thermoplastics. Reference to appropriate polymers that can be used in the facet joint implant sizing tool can be made to the following documents, all of which are incorporated herein by reference. These documents include: PCT Publication WO 02/02158 A1, dated Jan. 10, 2002, entitled “Bio-Compatible Polymeric Materials”; PCT Publication WO 02/00275 A1, dated Jan. 3, 2002, entitled “Bio-Compatible Polymeric Materials; and, PCT Publication WO 02/00270 A1, dated Jan. 3, 2002, entitled “Bio-Compatible Polymeric Materials.” Other materials such as Bionate®, polycarbonate urethane, available from the Polymer Technology Group, Berkeley, Calif., may also be appropriate because of the good oxidative stability, biocompatibility, mechanical strength and abrasion resistance. Other thermoplastic materials and other high molecular weight polymers can be used.

Selectively the tool for insertion can be manufactured from titanium, stainless steel or other materials suitable for insertion into the body.

The foregoing description of the invention has been presented for illustrative purposes. It is not intended to be exhaustive or to limit the invention to the precise forms disclosed. Many modifications and variations will be apparent to practitioners skilled in this art. The embodiments were chosen and described in order to explain the principles of the invention and its practical application, thereby enabling others skilled in the art to understand the invention for various embodiments and with various modifications as are suited to the particular use contemplated. It is intended that the scope of the invention be defined by the following claims and their equivalents.

What is claimed:

1. A tool comprising:
a spacer shaped similar to a cervical facet joint implant;
a handle; and
a pivot, wherein the distal end of the handle is connected with the proximal end of the spacer, allowing the base to pivot about the handle.

2. The tool of claim 1, wherein the spacer is adapted for sizing the cervical facet joint.

3. The tool of claim 2, where the thickness of the spacer varies among different spacers, wherein the said different spacers are used for sizing the cervical facet joint.

4. The tool of claim 1, further comprising a base, wherein the base contains one or more holes, where the holes are similar to holes in the cervical facet joint implant, wherein the proximal end of the base is connected to the handle, wherein the distal end of the base is connected with the proximal end of the spacer, allowing the spacer to pivot about the base.

5. The tool of claim 1, wherein the spacer is tapered to help the tool distract the facet joint.

6. The tool of claim 1, wherein the distal end of the joint spacer is rounded and is tapered in thickness to facilitate insertion into the cervical facet joint.

7. The tool of claim 1, wherein the spacer has one or more cleats adapted to be imbedded in the bone of the cervical facet joint.

8. A tool comprising:
a spacer;
a handle;
a pivot where the distal end of the handle is connected with the proximal end of the spacer allowing the spacer to pivot about the handle; and
a stop at the proximal end of the handle, the stop adapted to limit the insertion of the spacer.

9. The tool of claim 8, wherein the stop limits the rotation of the spacer relative to the handle.

10. A tool adapted to size a cervical facet joint in order to select a cervical facet joint implant for implanting in the cervical facet joint, said tool comprising:
a handle;
a spacer, wherein the spacer is shaped like a cervical artificial facet implant;
a pivot, the pivot connecting the proximal end of the spacer with the distal end of the handle; and
a stop at the proximal end of the base, the stop adapted to limit insertion of the spacer into the facet joint during sizing.

11. A method of sizing and or distracting a cervical facet joint comprising:
(a) accessing the cervical facet joint;
(b) selecting a tool, the tool having a spacer, a pivot, a handle and a stop, wherein the spacer pivots about the handle;
(c) inserting the tool into the cervical facet joint until the stop limits further insertion;
(d) pivoting the spacer away from the handle until it contacts the lamina;
(e) evaluating the fit of the tool, wherein evaluating includes evaluating the amount of distraction of the cervical facet joint;
(f) selecting one of a smaller and a larger tool depending on the measurement of step (e); and
(g) repeating steps (c)-(e) until a fit is found.

12. The method of claim 11, wherein the spacer is tapered, where in step (c) the tapering assists the tool distracting the facet joint.

13. The method as in claim 11, wherein the distal end of the spacer is rounded and is tapered in thickness, where in step (c) the tapering and roundedness facilitate insertion into the cervical facet joint.

14. The method as in claim 11, where the thickness of the spacer varies among different tools, where in steps (c)-(e) different tools are used for sizing the cervical facet joint.

15. The method of claim 11, wherein the spacer has one or more cleats, where in step (d) the cleats are adapted to be imbedded in the bone of the cervical facet joint to assist pivoting.

16. The method of claim 11, wherein the stop limits the rotation of the spacer relative to the handle.

17. The method of claim 11, wherein the tool further comprises a base, wherein the base contains one or more holes, where the one or more holes are similar to one or more holes in a cervical facet joint implant, where step (e) further comprises checking the position of the one or more holes in the base on the facet joint.
